(19) United States
(12) Patent Application Publication
(10) Pub. No.: US 2012/0289978 A1
(43) Pub. Date: Nov. 15, 2012

(54) RETAINING MECHANSIM

(75) Inventor: Clinton R. Jacob, Memphis, TN (US)

(73) Assignee: WARSAW ORTHOPEDIC, INC., Warsaw, IN (US)

(21) Appl. No.: 13/107,445

(22) Filed: May 13, 2011

(51) Int. Cl. A61B 17/00

(52) U.S. Cl. 606/151

(57) ABSTRACT

A retaining mechanism for use in affixing a stratum to bone is disclosed. The mechanism comprises a stratum and a retaining element. The retaining element comprises a central longitudinal axis, a stop end, a fastener end opposite that of the stop end, a first side, a second side, a first cut-out in the first side, a second cut-out in the second side, and a central cut-out. The stratum is configured to engage the retaining element such that when the retaining element is in a first position, the retaining element permits a fastener to be passed through a hole in the stratum, and when the retaining element is in a second position, the retaining element at least partially overlaps the hole so as to help prevent inadvertent backing out of the fastener after the fastener has been fully inserted into the hole.

### FIELD OF INVENTION

The present invention is directed to systems for affixing a stratum to bone.

### BACKGROUND

The present disclosure relates to retaining mechanisms, and more particularly, systems for affixing a stratum to bone.

### SUMMARY OF THE INVENTION

A retaining mechanism for use in affixing a stratum to bone is disclosed. The retaining mechanism comprises a stratum and a retaining element. The stratum comprises a first surface, a second surface opposing the first surface, and a hole extending between the first surface and the second surface, wherein the second surface is configured to engage at least a portion of the bone. The retaining element comprises a central longitudinal axis, a stop end situated at one end of the central longitudinal axis, a fastener end situated at an end of the central longitudinal axis opposite that of the stop end, a first side extending between the stop end and the fastener end, a second side extending between the stop end and the fastener end, an outer surface and an inner surface, a thickness defined by the distance between the outer surface and the inner surface, a length extending substantially along the central longitudinal axis between the stop end and the fastener end, a width extending substantially transverse to the central longitudinal axis and between the first side and the second side, a first cut-out in the first side extending a first distance toward the central longitudinal axis, a second cut-out in the second side extending a second distance toward the central longitudinal axis, and a central cut-out in the stop end extending a third distance toward the fastener end, wherein each of the cut-outs extends throughout the thickness of the retaining element at their respective locations. The inner surface is configured to engage with the first surface of the stratum, and the outer surface opposes the inner surface. The stratum is configured to engage the retaining element such that when the retaining element is in a first position, the retaining element permits a fastener to be passed through the hole, and when the retaining element is in a second position, the retaining element at least partially overlaps the hole so as to help prevent inadvertent backing out of the fastener after the fastener has been fully inserted into the hole.

Further, retaining mechanisms with various retaining elements are disclosed. In addition, a system for affixing stratum to bone is disclosed. The system comprises a retaining mechanism and at least one fastener configured to pass through the hole in the stratum and engage the bone.

### BRIEF DESCRIPTION OF THE DRAWINGS

FIG. 1 is an isometric top view of a retaining mechanism for affixing a stratum to bone;

FIG. 2 is an isometric top view of the retaining element of FIG. 1;

FIG. 3 is a cross-sectional isometric top view of the retaining mechanism of FIG. 1;

FIG. 4 is a cross-sectional isometric top view of the stratum of FIG. 1;

FIG. 5 is a top view of the retaining mechanism of FIG. 1;

FIG. 6 is an alternate design of a retaining element;

FIG. 7 is another alternate design of a retaining element.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

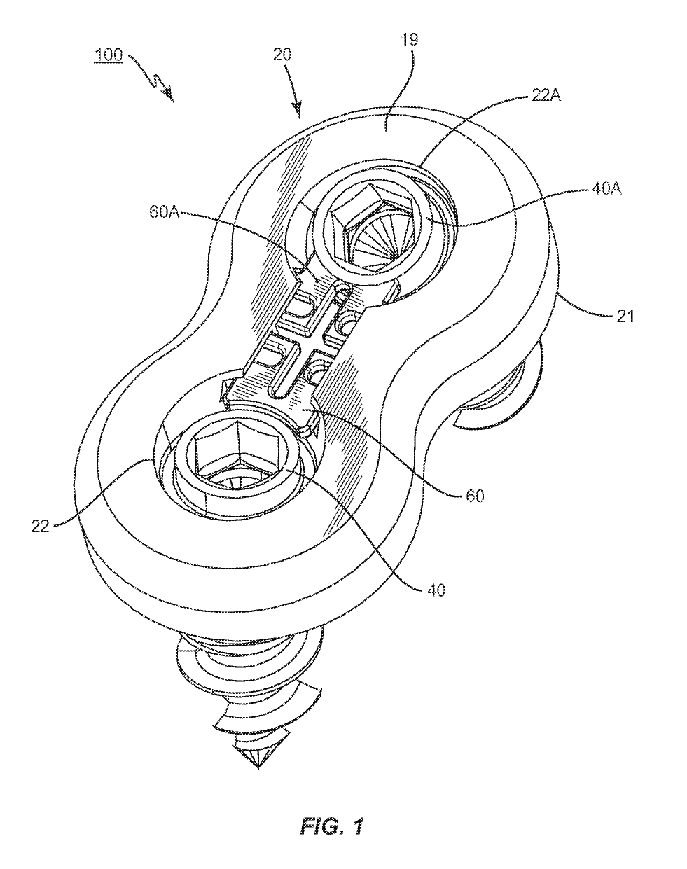

FIG. 1 shows an isometric top view of a retaining mechanism 100 for affixing a stratum 20 to bone, for example, to two or more levels of vertebral bodies. As shown in FIG. 1, the stratum 100 is designed for connecting two vertebral bodies (not shown), each vertebral body receiving one fastener 40 and 40A through holes 22 and 22A, respectively. As shown in FIG. 1, the stratum 20 may be, for example, a spinal plate for connecting cervical vertebrae by affixing the stratum 20 to, for example, the anterior surface of the vertebrae. Further, as shown in the Figures and as described herein, the fasteners 40 and 40A may be, for example, screws.

The retaining mechanism 100 comprises a stratum 20 and two retaining elements 60 and 60A. The stratum 20 comprises a first surface 19, a second surface 21 opposing the first surface 19, and a hole (for example, hole 22 or hole 22A) extending between the first surface 19 and the second surface 21, wherein the second surface 21 is configured to engage at least a portion of the bone.

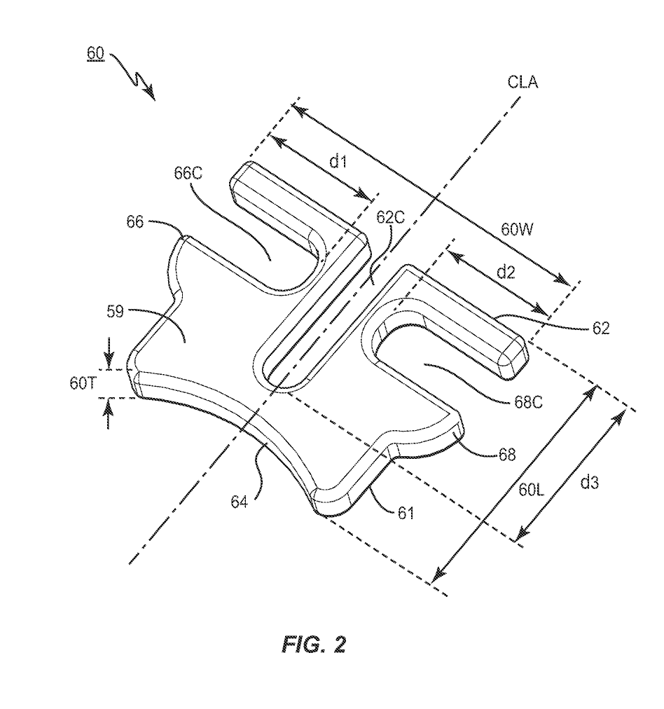

FIG. 2 shows an isometric top view of retaining element 60 of FIG. 1. As shown in FIG. 2, retaining element 60 comprises a central longitudinal axis CLA, a stop end 62 situated at one end of the central longitudinal axis CLA, a fastener end 64 situated at an end of the central longitudinal axis CLA opposite that of the stop end 62, a first side 66 extending between the stop end 62 and the fastener end 64, a second side 68 extending between the stop end 62 and the fastener end 64, an outer surface 59 and an inner surface 61, wherein the inner surface 61 is configured to engage with the first surface 19 of the stratum 20, and wherein the outer surface 59 opposes the inner surface 61, a thickness 60T defined by the distance between the outer surface 59 and the inner surface 61, a length 60L extending substantially along the central longitudinal axis CLA between the stop end 62 and the fastener end 64, a width 60W extending substantially transverse to the central longitudinal axis CLA and between the first side 66 and the second side 68, a first cut-out 66C in the first side 66 extending a first distance d1 toward the central longitudinal axis CLA, a second cut-out 68C in the second side 68 extending a second distance d2 toward the central longitudinal axis CLA, and a central cut-out 62C in the stop end 62 extending a third distance d3 toward the fastener end 64. Note that, as shown in the Figures, wherein each of the cut-outs 66C, 68C and 62C extend throughout the thickness 60T of the retaining element 60 at their respective locations.

The term “substantially” as used herein may be applied to modify any quantitative representation which could permissibly vary without resulting in a change in the basic function to which it is related. For example, and particularly if the retaining element 60 is not symmetric about the central longitudinal axis CLA, the length 60L may be considered to extend substantially along the central longitudinal axis CLA even if it is not exactly parallel to the central longitudinal axis CLA.

Further, as shown in FIG. 2, the stratum 20 is configured to engage the retaining element 60 such that when the retaining element is in a first position, the retaining element permits a fastener such as fastener 40 to be passed through the hole 22, and when the retaining element 60 is in a second position, the retaining element 60 at least partially overlaps the hole 22 so as to help prevent inadvertent backing out of the fastener 40 after the fastener 40 has been fully inserted into the hole 22.

As shown in FIGS. 1 and 2, the retaining element is flexible along the central longitudinal axis CLA such that when the fastener 40 is inserted into the hole 22, the retaining element 60 contracts and each cut-out contracts, thereby decreasing the length 60L of the retaining element 60, so as to allow the fastener 40 to enter the hole 22. After the fastener 40 has been fully inserted into the hole 22, the retaining element 60 expands and each cut-out returns to its original size or a size closer to its original size, thereby increasing its length 60L, so as to help prevent inadvertent backing out of the fastener 40.

As shown in the embodiment of FIG. 2, the third distance d3 extends more than half of the length 60L of the retaining element 60. In addition, as shown in the embodiment of FIG. 2, the first and second distances d1 and d2 each extend more than 25% of the width 60W of the retaining element 60 at their respective locations.

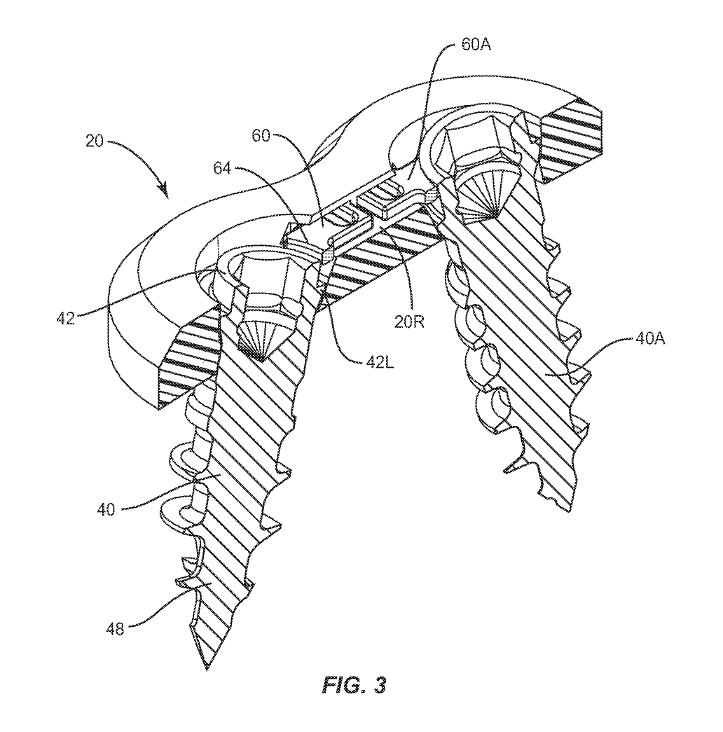

FIG. 3 shows a cross-sectional isometric top view of retaining mechanism 100 of FIG. 1, where the cross-section is taken along the central longitudinal axis CLA of the retaining element 60. As shown in FIG. 3, the stratum 20 further comprises a recess 20R configured to maintain the engagement of the retaining element 60 and the stratum 20. FIG. 3 shows the retaining mechanism 100 after the fasteners 40 and 40A have been fully inserted into the hole 22 and 22A, respectively. Specifically, as shown in FIG. 3, fastener 40 comprises a shaft 48 and a head 42. Further, as shown in FIG. 3, head 42 comprises a ledge 42L. When the ledge 40L abuts the fastener end 64 of the retaining element 60, the retaining element is in its first position, thereby permitting the fastener 40 to pass through the hole 22. As shown in FIG. 3, where the fastener 40 has been fully inserted into the hole 22, the retaining element 60 is in its second position, thereby partially overlapping the hole 22 so as to help prevent inadvertent backing out of the fastener 40. More specifically, as shown in FIG. 3, the fastener end 64 of the retaining element 60 at least partially overlaps the ledge 42 of the head 42 of fastener 40 so as to help prevent inadvertent backing out of the fastener 40.

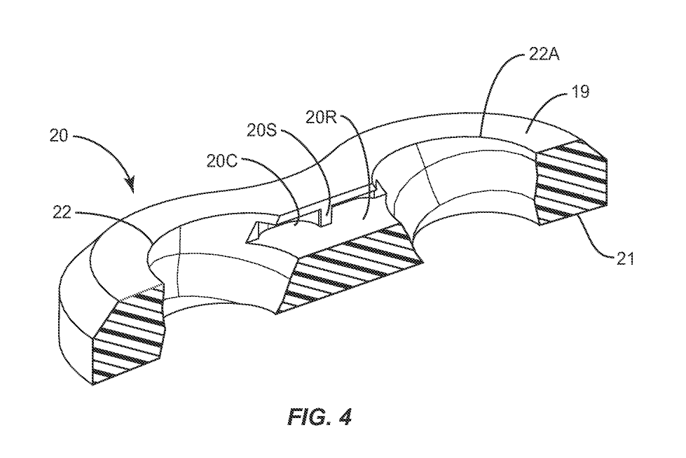

FIG. 4 shows a cross-sectional isometric top view of stratum 20 of retaining mechanism 100 of FIG. 1, where the cross-section is taken along the central longitudinal axis CLA of the stratum 20. In other words, FIG. 4 shows the stratum 20 of FIG. 3 with the retaining elements 60 and 60A and the fasteners 40 and 40A of FIG. 3 not shown. As shown in FIG. 4, the stratum 20 further comprises a first channel 20C and a second channel (not shown) for engaging the first side 66 and the second side 68 of the retaining element 60, respectively. In addition, as shown in FIG. 4, the stratum 20 further comprises a first stop 20S situated along the first channel 20C for engaging the first side 66 of the retaining element 60, and a second stop (not shown) situated along the second channel (not shown) for engaging the second side 68 of the retaining element 60, such that when, for example, fastener 40 is inserted into the hole 22, the retaining element 60 contracts because of forces exerted on the stop end 62 of the retaining element 60 by the first and second stops, respectively, and because of forces exerted on the fastener end 64 of the retaining element 60 by the fastener 40. Note that the second channel is similarly situated as the first channel 20C, but on the opposite side of the central longitudinal axis CLA. Similarly, the second stop is similarly situated as the first stop 20S, but on the opposite side of the central longitudinal axis CLA, that is, along the second channel.

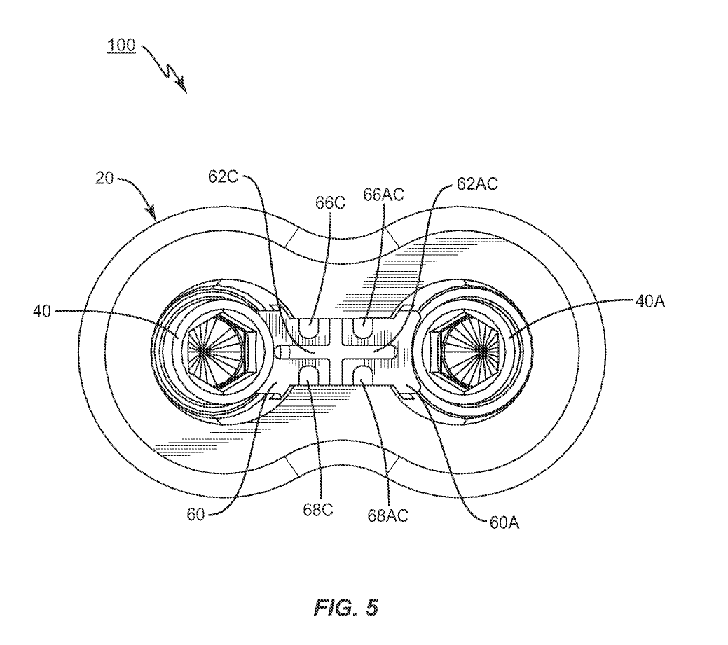

FIG. 5 shows a top view of the retaining mechanism 100 of FIG. 1. As shown in FIGS. 1 and 5, the retaining mechanism 100 comprises the stratum 20, a first retaining element 60 and a second retaining element 60A. Further, as shown in FIGS. 1 and 5, the stratum comprises a first hole 22 and a second hole 22A, and the second retaining element 60A has the same characteristics as that of the first retaining element 60 and is configured to engage with the second fastener 40A and the second hole 22A in the same manner that the first retaining element 60 is configured to engage with the first fastener 40 and first hole 22.

A system for affixing a stratum 20 to bone also is disclosed. The system comprises a retaining mechanism such as stratum 20 and retaining element 60, and a fastener such as fastener 40 configured to pass through the hole 22 and engage the bone. As shown in the embodiment of FIGS. 1 and 5, such a system comprises stratum 20, a first retaining element 60, a second retaining element 60A, and two fasteners 40 and 40A.

Also, FIG. 5 shows the first cut-out 66C in the first side 66 of the retaining element 60, as well as the second cut-out 68C in the second side 68 and the central cut-out 62C in the stop end 62 of the retaining element 60. Further, FIG. 5 shows the first cut-out 66AC in the first side of the retaining element 60A, as well as the second cut-out 68AC in the second side and the central cut-out 62AC in the stop end of the retaining element 60A.

Note that the shapes, sizes and arrangements of the retaining mechanisms disclosed herein may vary depending on design requirements or desired specifications. As long as the function of the retaining element 60 can be accomplished, numerous variations are possible. For example, a retaining element need not have the same shape as the retaining element shown in FIG. 2.

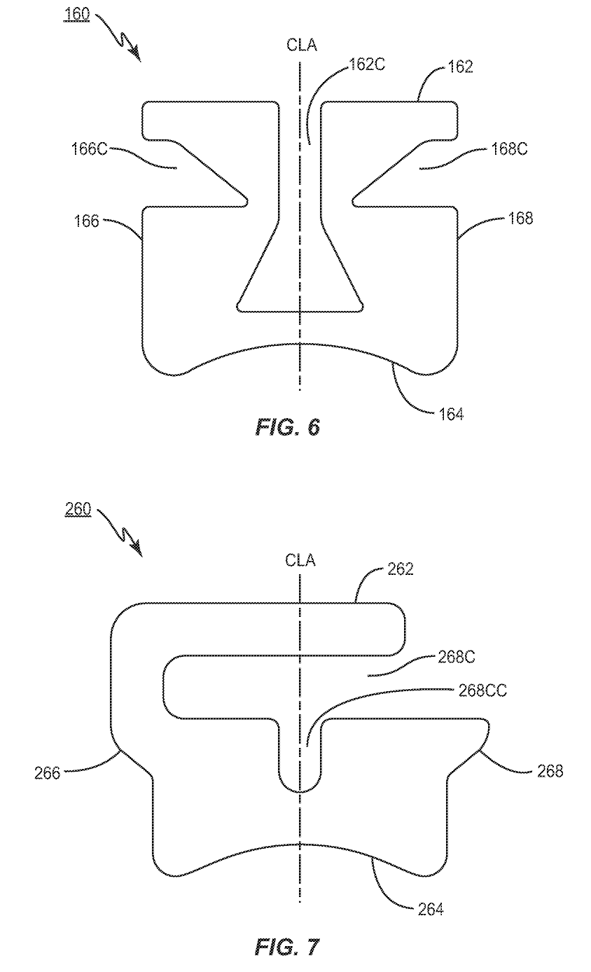

FIG. 6 shows merely one alternate design of such a retaining element. Specifically, FIG. 6 shows a retaining element 160 comprising a first side 166, a second side 168, a fastener end 164 and a stop end 162. Further, retaining element 160 comprises a first cut-out 166C in the first side 166, a second cut-out in the second side 168 and a central cut-out 162C in the stop end 162. Note that although the first and second sides 166 and 168 of retaining element 160 and their respective cut-outs are symmetrical about the central longitudinal axis CLA, this need not be the case. For example, the second cut-out 168C may have a different shape than that of the first cut-out 166C and/or the central cut-out need not be symmetrical about the central longitudinal axis CLA. Further, the number of cut-outs need not be limited to those shown in FIG. 2 or FIG. 6. In addition, for example, a retaining element may have the same basic shape as retaining element 60, but the distances d1, d2 and d3 may vary from that shown in FIG. 2. Accordingly, as noted, the retaining element and/or corresponding stratum 30 may have shapes other than those shown as long as they satisfy the function described herein—being able to adequately engage with each other and a corresponding fastener.

FIG. 7 shows another alternate design of a retaining element 260. Specifically, FIG. 7 shows a retaining element 260 comprising a first side 266, a second side 268, a fastener end 264 and a stop end 262. Further, retaining element 260 comprises a side cut-out 268C in the second side 268, and the side cut-out 268C has a central cut-out 268CC. Note that retaining element 260 does not have a side cut-out in the first side 266 and the central cut-out is not in the stop end 262, but is in the side cut-out 268C. Accordingly, retaining element 260 is an example of a retaining element that has is not symmetrical about the central longitudinal axis CLA. Similarly, as shown in FIG. 7, the side cut-out 268C of retaining element 260 is not symmetrical about the central longitudinal axis CLA, the number of cut-outs of retaining element 260 is not the same as that shown in the retaining elements shown in FIG. 2 and FIG. 6. In addition, retaining element 260 has shapes other than those shown in previous figures, but satisfies the required function of being able to adequately engage with a stratum and a fastener. Further, as FIG. 7 helps to demonstrate, the central cut-out 268CC, for example, provides a benefit of improving ease of assembly. That is, central cut-out 268CC (as well as each of central cut-outs 62C and 162C) allows for some contraction/compression to occur in a direction substantially perpendicular to the central longitudinal axis CLA. In doing so, as shown in the example of FIG. 7, the retaining element 260 will be able to reduce its width so as to make it easier for insertion of the retaining element 260 into a stratum, or more specifically, for insertion of the sides 266 and 268 of retaining element 260 into channels in a recess of a stratum such as stratum 20.

In the embodiments shown and described herein, the stratum 20 is substantially rigid. Accordingly, parts of the stratum 20 such as channel 20C and stop 20S are substantially rigid. Further, in the embodiments described herein, the stratum may be made of a variety of biocompatible materials (metal or non-metal), including but not limited to, Titanium Alloys, commercially available Titanium, stainless steel, polyetheretherketone (“PEEK”), cobalt chrome (“CoCr”), polyetherketoneketone (“PEKK”), ultra high molecular weight polyethylene (“UHMWPE”), polyethylene, shape memory metals, other polymers or any combination of such materials. Similarly, retaining mechanisms and/or the fasteners may be made of the same materials. Also, any suitable materials know in the art may work for each of these elements as well as for other elements described herein.

In addition, in the embodiments shown, each retaining element has elastic properties. Thus, a retaining element may comprise a material that has elastic properties. For example, the retaining element may comprise a material such as metal that is elastic. In addition, the retaining element, for example, may be made of Nickel Titanium (NiTi), commercially pure Titanium, a Titanium alloy or any combination of such materials.

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. Furthermore, as used herein, the terms and components may be interchanged. It is understood that all spatial references, such as “first,” “second,” “superior,” “inferior,” “anterior,” “posterior,” “outer,” “inner,” “upper,” “underside,” “top,” “bottom,” and “perimeter” are for illustrative purposes only and can be varied within the scope of the disclosure.

1. A retaining mechanism for use in affixing a stratum to bone, the mechanism comprising:
a stratum comprising a first surface, a second surface opposing the first surface, and a hole extending between the first surface and the second surface, wherein the second surface is configured to engage at least a portion of the bone, and
a retaining element comprising:
a central longitudinal axis;
a stop end situated at one end of the central longitudinal axis;
a fastener end situated at an end of the central longitudinal axis opposite that of the stop end;
a first side extending between the stop end and the fastener end;
a second side extending between the stop end and the fastener end;
an outer surface and an inner surface, wherein the inner surface is configured to engage with the first surface of the stratum, and wherein the outer surface opposes the inner surface;
a thickness defined by the distance between the outer surface and the inner surface;
a length extending substantially along the central longitudinal axis between the stop end and the fastener end;
a width extending substantially transverse to the central longitudinal axis and between the first side and the second side;
a first cut-out in the first side extending a first distance toward the central longitudinal axis;
a second cut-out in the second side extending a second distance toward the central longitudinal axis; and
a central cut-out in the stop end extending a third distance toward the fastener end, wherein each of the cut-outs extends throughout the thickness of the retaining element at their respective locations;
wherein the stratum is configured to engage the retaining element such that:
when the retaining element is in a first position, the retaining element permits a fastener to be passed through the hole; and
when the retaining element is in a second position, the retaining element at least partially overlaps the hole so as to help prevent inadvertent backing out of the fastener after the fastener has been fully inserted into the hole.

2. The mechanism of claim 1, wherein the retaining element is flexible along the central longitudinal axis such that:
when a fastener is inserted into the hole, the retaining element contracts, thereby decreasing its length, so as to allow the fastener to enter the hole; and
after the fastener has been fully inserted into the hole, the retaining element expands, thereby increasing its length, so as to help prevent inadvertent backing out of the fastener.

3. The mechanism of claim 2, wherein the third distance extends more than half of the length of the retaining element.

4. The mechanism of claim 3, wherein the first and second distances each extend more than 25% of the width of the retaining element at their respective locations.

5. The mechanism of claim 2, wherein the stratum further comprises a recess configured to maintain the engagement of the retaining element and the stratum.

6. The mechanism of claim 5, wherein the stratum further comprises a first channel and a second channel for engaging the first side and the second side of the retaining element, respectively.

7. The mechanism of claim 6, wherein the stratum further comprises a first stop situated along the first channel for engaging the first side of the retaining element, and a second stop situated along the second channel for engaging the second side of the retaining element, such that when the fastener is inserted into the hole, the retaining element contracts because of forces exerted on the stop end of the retaining element by the first and second stops, respectively, and because of forces exerted on the fastener end of the retaining element by the fastener.

8. The mechanism of claim 1, wherein the retaining element comprises material having elastic properties.

9. The mechanism of claim 1, wherein:
the hole is a first hole;
the retaining element is a first retaining element;
the stratum further comprises a second hole; and
the retaining mechanism further comprises a second retaining element with the same characteristics of the first retaining element and configured to engage with the second fastener and the second hole in the same manner that the first retaining element is configured to engage with the first fastener and first hole.

10. A system for affixing the stratum of claim 1 to the bone, the system comprising:
the retaining mechanism; and
a fastener configured to pass through the hole and engage the bone.

11. A retaining mechanism for use in affixing a stratum to bone, the mechanism comprising:
a stratum comprising a first surface, a second surface opposing the first surface, and a hole extending between the first surface and the second surface, wherein the second surface is configured to engage at least a portion of the bone, and
a retaining element comprising:
a central longitudinal axis;
a stop end situated at one end of the central longitudinal axis;
a fastener end situated at an end of the central longitudinal axis opposite that of the stop end;
a first side extending between the stop end and the fastener end;
a second side extending between the stop end and the fastener end;
an outer surface and an inner surface, wherein the inner surface is configured to engage with the first surface of the stratum, and wherein the outer surface opposes the inner surface;
a thickness defined by the distance between the outer surface and the inner surface;
a length extending substantially along the central longitudinal axis between the stop end and the fastener end;
a width extending substantially transverse to the central longitudinal axis and between the first side and the second side;
a first cut-out in the first side extending a first distance toward the central longitudinal axis;
a second cut-out in the second side extending a second distance toward the central longitudinal axis; and
a central cut-out in the stop end extending a third distance toward the fastener end, wherein each of the cut-outs extends throughout the thickness of the retaining element at their respective locations;
wherein the stratum is configured to engage the retaining element such that:
when the retaining element is in a first position, the retaining element permits a fastener to be passed through the hole; and
when the retaining element is in a second position, the retaining element at least partially overlaps the hole so as to help prevent inadvertent backing out of the fastener after the fastener has been fully inserted into the hole;
wherein the retaining element is flexible along the central longitudinal axis such that:
when a fastener is inserted into the hole, the retaining element contracts, thereby decreasing its length, so as to allow the fastener to enter the hole; and
after the fastener has been fully inserted into the hole, the retaining element expands, thereby increasing its length, so as to help prevent inadvertent backing out of the fastener; and
wherein the third distance extends more than half of the length of the retaining element; the first and second distances each extend more than 25% of the width of the retaining element at their respective locations.

12. The mechanism of claim 11, wherein the stratum further comprises a recess configured to maintain the engagement of the retaining element and the stratum.

13. The mechanism of claim 12, wherein the stratum further comprises a first channel and a second channel for engaging the first side and the second side of the retaining element, respectively.

14. The mechanism of claim 11, wherein the stratum further comprises a first stop situated along the first channel for engaging the first side of the retaining element, and a second stop situated along the second channel for engaging the second side of the retaining element, such that when the fastener is inserted into the hole, the retaining element contracts because of forces exerted on the stop end of the retaining element by the first and second stops, respectively, and because of forces exerted on the fastener end of the retaining element by the fastener.

15. The mechanism of claim 11, wherein the retaining element comprises material having elastic properties.

16. A system for affixing the stratum of claim 11 to the bone, the system comprising:
the retaining mechanism; and
a fastener configured to pass through the hole and engage the bone.

17. A retaining mechanism for use in affixing a stratum to bone, the mechanism comprising:
a stratum comprising a first surface, a second surface opposing the first surface, and a hole extending between the first surface and the second surface, wherein the second surface is configured to engage at least a portion of the bone, and
a retaining element comprising:
a central longitudinal axis;
a stop end situated at one end of the central longitudinal axis;
a fastener end situated at an end of the central longitudinal axis opposite that of the stop end;
a first side extending between the stop end and the fastener end;
a second side extending between the stop end and the fastener end;
an outer surface and an inner surface, wherein the inner surface is configured to engage with the first surface of the stratum, and wherein the outer surface opposes the inner surface;
a thickness defined by the distance between the outer surface and the inner surface;
a length extending substantially along the central longitudinal axis between the stop end and the fastener end;
a width extending substantially transverse to the central longitudinal axis and between the first side and the second side;
a first cut-out in the first side extending a first distance toward the central longitudinal axis;
a second cut-out in the second side extending a second distance toward the central longitudinal axis; and
a central cut-out in the stop end extending a third distance toward the fastener end, wherein each of the cut-outs extends throughout the thickness of the retaining element at their respective locations;
wherein the stratum is configured to engage the retaining element such that:
when the retaining element is in a first position, the retaining element permits a fastener to be passed through the hole; and
when the retaining element is in a second position, the retaining element at least partially overlaps the hole so as to help prevent inadvertent backing out of the fastener after the fastener has been fully inserted into the hole;
wherein the retaining element is flexible along the central longitudinal axis such that:
when a fastener is inserted into the hole, the retaining element contracts, thereby decreasing its length, so as to allow the fastener to enter the hole; and
after the fastener has been fully inserted into the hole, the retaining element expands, thereby increasing its length, so as to help prevent inadvertent backing out of the fastener;
wherein the third distance extends more than half of the length of the retaining element; the first and second distances each extend more than 25% of the width of the retaining element at their respective locations; and
wherein the stratum further comprises a first stop for engaging the first side of the retaining element, and a second stop for engaging the second side of the retaining element, such that when the fastener is inserted into the hole, the retaining element contracts because of forces exerted on the stop end of the retaining element by the first and second stops, respectively, and because of forces exerted on the fastener end of the retaining element by the fastener.

18. The mechanism of claim 17, wherein the stratum further comprises a recess configured to maintain the engagement of the retaining element and the stratum.

19. The mechanism of claim 18, wherein the stratum further comprises a first channel and a second channel for engaging the first side and the second side of the retaining element, respectively, and the first stop is situated along the first channel and the second stop is situated along the second channel.

20. A system for affixing the stratum of claim 17 to the bone, the system comprising:
the retaining mechanism; and
a fastener configured to pass through the hole and engage the bone.
